(19) United States
(12) Patent Application Publication
(10) Pub. No.: US 2009/0274710 A1
(43) Pub. Date: Nov. 5, 2009

(54) TRANS-MEMBRANE-ANTIBODY INDUCED INHIBITION OF APOPTOSIS

(75) Inventors: Heinz Kohler, Lexington, KY (US); Sybille Muller, Lexington, KY (US); Thomas L. Brown, Beavercreek, OH (US); Yunfeng Zhao, Lexington, KY (US); A. Charles Morgan, JR., Scottsdale, AZ (US)

Correspondence Address: GIFFORD, KRASS, SPRINKLE,ANDERSON & CITKOWSKI, P.C, PO BOX 7021, TROY, MI, 48007-7021, US

(73) Assignee: InNexus Biotechnology International Limited, Scottsdale, AZ (US)

(21) Appl. No.: 12/421,398

(22) Filed: Apr. 9, 2009

Related U.S. Application Data
(63) Continuation of application No. 10/795,081, filed on Mar. 5, 2004, now abandoned.
Application No. 10/795,081 is a continuation-in-part of application No. 09/865,281, filed on May 29, 2001, now abandoned.
Application No. 09/865,281 is a continuation-in-part of application No. 09/070,907, filed on May 4, 1998, now Pat. No. 6,238,667.
(60) Provisional application No. 60/451,980, filed on Mar. 5, 2003.
(60) Provisional application No. 60/059,515, filed on Sep. 19, 1997.

(51) Int. Cl. A61K 39/395; C07K 16/18

(52) U.S. Cl. 424/178.1; 530/391.1

(57) ABSTRACT

Cell suicide (apoptosis) is associated with pathogenesis, for example, it is the major cause for the loss of neurons in Alzheimer's disease. Caspase-3 is critically involved in the pathway of apoptosis. Superantibody (SAT)-trans-membrane technology has been used to produce antibodies against the caspase enzyme in an effort to inhibit apoptosis in living cells. The advantage of using trans-membrane antibodies as apoptosis inhibitors is their specific target recognition in the cell and their lower toxicity compared to conventional apoptosis inhibitors. It is shown that a MTS-transport-peptide modified monoclonal anti-caspase-3 antibody reduces actinomycin D-induced apoptosis and cleavage of spectrin in living cells. These results indicate that antibodies conjugated to a membrane transporter peptide have a therapeutic potential to inhibit apoptosis in a variety of diseases.

### CROSS REFERENCE TO RELATED APPLICATIONS

The present application is a continuation of U.S. patent application Ser. No. 10/795,081, filed Mar. 5, 2004, which is a continuation-in-part of U.S. application Ser. No. 09/865,281, filed May 29, 2001, now abandoned, which is a continuation-in-part of U.S. patent application Ser. No. 09/070,907, filed May 4, 1998, now U.S. Pat. No. 6,238,667, which claims priority from U.S. Provisional Application Ser. No. 60/059,515, filed Sep. 19, 1997.

U.S. patent application Ser. No. 10/795,081 also claims the benefit of U.S. Provisional Application No. 60/451,980, filed Mar. 5, 2003. The entire content of each patent and patent application is incorporated herein by reference.

### FIELD OF THE INVENTION

The present invention relates to fusion proteins comprising whole biologically active peptides and antibodies, or fragments thereof. Specifically, the fusion proteins of the present invention combine the molecular recognition of antibodies with a biological activity such as immuno-stimulatory activity, membrane transport activity, and homophilic activity. The present invention further relates to fusion proteins having the binding properties of an antibody and including a biologically active peptide sequence flanked by loop forming or other conformation-conferring sequences so as to constrain the conformational flexibility of the biologically active peptide and to increase its affinity for its biological target. The present invention also relates to the use of antibodies and conjugates thereof in the inhibition of programmed cell death, i.e., apoptosis.

### BACKGROUND OF THE INVENTION

Antibodies have been praised as “magic bullets” to combat disease; however, the promises made for antibodies have never been fully realized. This is due in part to the fact that antibodies represent only one arm of the immune defense, where T-cells provide the other strategy in immune defense. However, antibodies are ideal targeting and delivery devices. They are adapted for long survival in blood, have sites that help vascular and tissue penetration, and are functionally linked with a number of the defense mechanisms of innate immunity. One such mechanism is the complement system, which helps to destroy pathogens and is involved in the regulation of immune responses. For example, the complement fragment C3d binds to the CR2 receptor on B-cells, which is also the binding site for Epstein-Barr virus. Binding of Epstein-Barr virus to CR2 activates B-cells. Accumulated evidence has shown that the CR2 receptor (CD19/Cd20/CD81 complex) has an immuno-stimulatory role and is activated by C3d.

Monoclonal antibodies have been developed for many therapeutic uses. For example, diseases currently targeted by monoclonal antibodies include heart conditions, cancers, neurological defects and autoimmune diseases. Virtually all of these current therapeutic uses rely on the inherent therapeutic efficacy of the particular monoclonal antibodies, such as with the drugs HERCEPTIN and RITUXAN. Since most monoclonal antibodies do not express such inherent therapeutic activity, development has focused on the addition of therapeutic properties by conjugation of a variety of different toxic agents, such as protein toxins or their subunits, drugs currently used in the chemotherapeutic treatment of cancer, drugs which failed to progress in clinical development due to unacceptable toxicity, or radioisotopes.

To make such conjugates effective, a monoclonal antibody delivering such toxic agents must be able to bind to its target antigen and internalize into cells to carry the toxic agent inside where it can be effective at damaging DNA or inhibiting protein synthesis or other metabolic functions of the targeted cell. Few antibodies inherently express such a property—the ones that do produce very potent immunoconjugates. As such, screening assays have been developed to test for such antibodies but few antibodies have been identified that combine this quality with an appropriate targeting specificity.

There have been other approaches to instill internalizing ability into an antibody. Whole protein toxins which combine an active subunit with a cell binding subunit are effective in enhancing internalization when conjugated to an antibody but oftentimes reduce the selectivity of the antibody thereby leading to potential toxicity. Lipophilic drugs have also been used to enhance internalization and intracellular delivery in conjugated form but as with toxins will also reduce the selectivity of a conjugate. Other methods have been used to permeabilize or by microinjection allow better entry into cells. Both of these methods have serious drawbacks. Permeabilization of cells, e.g., by saponin, bacterial toxins, calcium phosphate, electroporation, etc., can only be practically used for ex vivo methods, and these methods cause damage to the cells. Microinjection requires highly skilled technicians (thus limiting its use to a laboratory setting), it physically damages the cells, and it has only limited applications as it cannot be used to treat, for example, a mass of cells or an entire tissue, because one cannot feasibly inject large numbers of cells.

Another example of how antibodies can be used to enhance the immune response has been demonstrated by the work of Zanetti and Bona (Zanetti, M., Nature, 355: 466-477, 1992; Zaghouani H.; Anderson S. A., Sperbeer K. E., Daian C. Kennedy R. C., Mayer L. and Bona C. A., Proc. Nat. Acad. Science USA, 92: 631-635, 1995). These authors have replaced the CDR3 sequence of the Ig heavy chain with a sequence resembling T-cell and B-cell antigens (epitopes) using molecular biology methods and have shown that these modified antibodies induce potent immune response specific for the inserted groups.

The biological properties of the antibodies can be enhanced with respect to overall avidity for antigen and the ability to penetrate cellular and nuclear membranes. Antigen binding is enhanced by increasing the valency of antibodies such as in pentameric IgM antibodies. Valency and avidity are also increased in certain antibodies that are self-binding or homophilic (Kang, C. Y., Cheng, H. L., Rudikoff, S. and Kohler, H., J. Exp. Med. 165:1332, 1987; Xiyun, A. N., Evans, S. V., Kaminki, M. J., Fillies, S. F. D., Resifeld, R. A., Noughton, A. N. and Chapman, P. B., J. Immunol. 157: 1582-1588, 1996). A peptide in the heavy chain variable region was identified which inhibited self-binding (Kano, C. Y. Brunck, T. K., Kieber-Emmons, T., Blalock, J. E. and Kohler, H., Science, 240: 1034-1036, 1988). The insertion of a self-binding peptide sequence into an antibody endows the property of self-binding and increases the valency and overall avidity for the antigen.

Similarly, the addition of a signal peptide to antibodies facilitates transmembrane transport as demonstrated by Rojas et al, Nature Biotechnology, 16: 370-375 (1998). Rojas et al. have generated a fusion protein containing a 12-mer peptide and have shown that this protein has cell membrane permeability.

Signal peptide sequences that express the common motif of hydrophobicity mediate translocation of most intracellular secretory proteins across mammalian endoplasmic reticulum (ER) and prokaryotic plasma membranes through the putative protein-conducting channels. The major model implies that the proteins are transported across membranes through a hydrophilic protein-conducting channel formed by a number of membrane proteins. In eukaryotes, newly synthesized proteins in the cytoplasm are targeted to the ER membrane by signal sequences that are recognized generally by the signal recognition particle (SRP) and its ER membrane receptors. This targeting step is followed by the actual transfer of protein across the ER membrane and out of the cell through the putative protein-conducting channel. Signal peptides can also interact strongly with lipids, supporting the proposal that the transport of some secretory proteins across cellular membranes may occur directly through the lipid bilayer in the absence of any proteinaceous channels. Such signal peptides can be used to enhance internalization of antibodies or other biologically active molecules into cells and are the subject of several patents (U.S. Pat. Nos. 5,807,746, No. 6,043,339 and No. 6,238,667).

Antibodies have been used as delivery devices for several biologically active molecules, such as toxins, drugs and cytokines. Often fragments of antibodies, Fab or scFv, are preferred because of better tissue penetration and reduced “stickiness”.

There are two practical methods for attaching molecules, such as peptides, to antibody molecules. One method is to use chemical crosslinking, such as the affinity-crosslinking method described in U.S. Ser. No. 09/070,907. Another method is to design a fusion gene containing DNA encoding the antibody and the peptide and to express the fusion gene, which method is the subject of the present application.

Antibody fusion proteins are typically engineered with entire genes of large proteins or domains of such proteins that afford a biological function. Previous small peptide-antibody fusion proteins have typically been made mainly for the purpose of facilitating purification or characterization of the antibody.

Methods of creating fusion proteins are described, for example, in the following U.S. patents, the pertinent disclosures of which are incorporated herein by reference: U.S. Pat. No. 5,563,046 to Mascarenhas et al; U.S. Pat. No. 5,645,835 to Fell, Jr.; U.S. Pat. No. 5,668,225 to Murphy; U.S. Pat. No. 5,698,679 to Nemazee; U.S. Pat. No. 5,763,733 to Whitlow et al; U.S. Pat. No. 5,811,265 to Querteimous et al; U.S. Pat. No. 5,908,626 to Chang et al; U.S. Pat. No. 5,969,109 to Bona et al; U.S. Pat. No. 6,008,319 to Epstein et al; U.S. Pat. No. 6,117,656 to Seed; U.S. Pat. No. 6,121,424 to Whitlow et al; U.S. Pat. No. 6,132,992 to Ledbetter et al; U.S. Pat. No. 6,207,804 to Huston et al; and U.S. Pat. No. 6,224,870 to Segal. Methods of creating Ig fusion proteins are described, for example, in Antibody Engineering, 2nd ed. ed.: Carl A. K. Borrebaeck, Oxford University Press 1995, and in Molecular Cloning: A Laboratory Manual, 2.sup.nd ed., Cold Spring Harbor Press, 1989, the pertinent disclosures of which are incorporated herein by reference.

Fusion proteins including those with immunoglobulins primarily incorporating active domains of proteins such as cytokines, toxins, enzymes, etc. with targeting domains of immunoglobulins including the CDR's (complementarity-determining regions) and other variable regions and domains not directly involved in antigen binding but through secondary interactions able to confer increased affinity of binding are described, for example, in the following publications incorporated herein by reference:
Guo L; Wang J; Qian S; Yan X; Clien R; Meng G, “Construction and structural modeling of a single-chain Fv-asparaginase fusion protein resistant to proteolysis.” Biotechnol. Bioeng., 2000 Nov. 20; 70(4):456-63.
Muller B H; Chevrier D; Boulain J C; Guesdon J L “Recombinant single-chain Fv antibody fragment-alkaline phosphatase conjugate for one-step immunodetection in molecular hybridization.” J. Immunol. Methods 1999 Jul. 30; 227(1-2):177-85.
Griep R A; van Twisk C; Kerschbaumer R J; Harper K; Torrance L; Himmler G; van der Wolf J M; Schots “pSKAP/S: An expression vector for the production of single-chain Fv alkaline phosphatase fusion proteins.” Protein Expr. Purif 1999 June; 16(11):63-9.
Vallera D A; Panoskaltsis-Mortari A; 1 C; Ramakrishnan S; Eide C R; Kreitman R J; Nicholls P J; Pennell C; Blazar B R “Anti-graft-versus-host disease effect of DT390-anti-CD3sFv, a single-chain Fv fusion immunotoxin specifically targeting the CD3 epsilon moiety of the T-cell receptor.” Blood 1996 Sep. 15; 88(6):2342-53.
Gupta S; Eastman J; Silski C; Ferkol T; Davis P B “Single chain Fv: a ligand in receptor-mediated gene delivery.” Gene Ther. 2001 April; 8(8):586-92.
Goel A; Colcher D; Koo J S; Booth B J; Pavlincova G; Batra “Relative position of the hexahistidine tag effects binding properties of a tumor-associated single-chain Fv construct.” Biochim Biophys Acta 2000 Sep. 1; 1523(1):13-20.

Fusion proteins designed to have biological activity may be constructed using linear peptide sequences derived from a whole biologically active protein. However, such peptides have typically lower affinity than the entire protein. Since the incorporation of a peptide into a fusion protein is less cumbersome than the incorporation of an entire functional protein, there is a need for fusion proteins containing peptides living a binding affinity as good as a full-length protein.

The present invention also relates to the use of antibodies and fragments thereof in the inhibition of apoptosis. Cell suicide (apoptosis) is a mechanism used beneficially by living organisms in cell differentiation in organ development and elimination of damaged cells. However, apoptosis can also be associated with forms of pathogenesis. For example, it is the major cause for the loss of neurons in Alzheimer's disease and tissue loss during myocardial infarction. Also, T lymphocytes from HIV-1 infected individuals undergo spontaneous apoptosis in the absence of a stimulus compared to uninfected T cells cultured under the same conditions. The “spontaneous apoptosis” of CD4+ and CD8+ cells has been shown to be accelerated by the in-vitro addition of an HIV-1 related, anti-idiotypic antibody.

Caspase enzymes, e.g., caspase-3, are critically involved in the pathway of apoptosis. A number of materials and methods have been proposed for inhibiting caspase action in an effort to inhibit apoptosis. For example, U.S. Pat. No. 6,566,338 (Weber et al.) proposes the use of caspase inhibitors generally for treating, ameliorating, and preventing non-cancer cell death during chemotherapy and radiation therapy and for treating and ameliorating the side effects of chemotherapy and radiation therapy of cancer. U.S. Pat. No. 6,596,693 (Keana et al.) reports that certain dipeptides can be potent inhibitors of apoptosis. U.S. Pat. Nos. 6,689,784 (Bebbington, et al.) and 6,620,782 (Cai et al.) propose a class of carbamates and substituted 2-aminobenzamides, respectively, as inhibitors of apoptosis. Also, U.S. Pat. No. 6,426,413 (Wannamaker et al.) is a representative proposal for a class of caspase inhibitors called interleukin-1beta-converting enzyme inhibitors. Additionally, U.S. Pat. No. 6,228,603 (Reed et al.) proposes a screening assay for identifying agents that alter the specific association of an inhibitor of apoptosis with a caspase, such as caspase-3 or caspase-7.

Yet another novel approach for inhibiting caspase enzymes involves the use of so-called “Superantibody Technology (SAT)”. See, e.g., WO 02/097041, entitled “Fusion Proteins of Biologically Active Peptides and Antibodies” (co-assigned to Immpheron, Inc. and Innexus Corporation). One proposed application of SAT is the use of antibodies against caspase enzymes in order to inhibit apoptosis in living cells. For example, one aspect of the present invention contemplates intracellular delivery of an antibody or antibody fragment immunospecific for an enzyme involved in apoptosis. Some expected advantages of trans-membrane antibodies as apoptosis inhibitors are their specific target recognition in the cell and their lower toxicity compared to conventional apoptosis inhibitors. It is an object of the present invention to provide such membrane-penetrating antibodies for therapeutic benefit.

### SUMMARY OF THE INVENTION

The present invention provides a fusion protein comprising an antibody domain and a peptide domain, wherein the biological activity of the peptide domain is selected from the group consisting of immuno-stimulatory, membrane transport and homophilic activities. The peptide is covalently linked to a site on the antibody so that the incorporated peptide does not compromise the antigen recognition of the antibody. In the present invention, this is accomplished by a method comprising the steps of creating a fusion gene comprising a nucleic acid sequence encoding an antibody and a nucleic acid sequence encoding the peptide, wherein the nucleic acid sequence encoding the peptide is located inside the nucleic acid sequence encoding the antibody at a site wherein, when the fusion is expressed, the fusion protein that is created thereby includes the antibody plus the peptide, and the peptide is connected to the antibody at a site that does not interfere with antigen binding of the antibody, and expressing the fusion gene to create the fusion protein. In particular, the fusion protein may be created by providing a gene encoding an antibody, wherein the gene is mutated to contain a restriction site, wherein the restriction site is located away from any section of the gene that encodes an antigen-binding site of the antibody, inserting a DNA sequence encoding a peptide having a biological activity selected from the group consisting of immuno-stimulatory, membrane transport and homophilic activities into restriction site of the gene encoding the antibody to create a fusion gene, and wherein the DNA sequence encoding the peptide is inserted so that it is in-frame with the gene encoding the antibody, and expressing the fusion gene to create a fusion protein.

In order to enhance the biological activity of the peptide, the peptide may be flanked by loop-forming or conformation-conferring sequences.

The invention also provides a composition and a pharmaceutical composition comprising a fusion protein of a peptide having a biological activity selected from the group consisting of immuno-stimulatory, membrane transport and homophilic activities and an antibody.

The invention of creating fusion proteins of biologically active peptides and antibodies includes peptides which comprise self-binding, stimulate lymphocytes and allow transport across biological membranes.

A further aspect of the present invention is for novel compounds and methods for regulating cell function, either in normal or infected cells. In particular, such compounds and methods entail the use of an antibody, or antibody fragment thereof, conjugated to a membrane transporter peptide. The antibody, or fragment thereof, is preferably immunospecific, i.e., it recognizes and binds specifically with high affinity to, for such protein targets as: (a) signaling proteins internal the cell, such as caspases, kinases, and phosphatases, (b) immature virion proteins prior to intracellular assembly, (c) cell-surface or intracellular tumor antigens, (d) nuclear or nucleolar proteins that are involved in regulation of DNA synthesis and gene expression, or (e) cytoskeletal proteins that participate in cell proliferation or cytostasis. Either polyclonal or monoclonal antibodies can be used.

In a preferred aspect of the invention, an aforementioned compound is effective in inhibiting apoptosis and comprises an anti-caspase antibody, or fragment thereof, conjugated to a membrane transporter peptide. A particularly preferred antibody is an anti-caspase-3 antibody.

In a second preferred aspect of the invention, an aforementioned membrane transporter peptide is a translocation sequence (MTS) peptide, such as one endogenous to Kaposi fibroblast factor, TAT peptides of HIV-1, antennapedia homeodomain-derived peptide, herpes virus protein VP22, or transportan peptide. A particularly preferred MTS peptide comprises the amino acid residue sequence AAVLLPVLLAAP (SEQ ID NO: 9), such as the peptide sequence KGEGAAVLLPVLLAAPG (SEQ ID NO: 8).

Also contemplated is a pharmaceutical composition effective in inhibiting apoptosis in human cells, and which therefore is implicated as being effective in the treatment of human diseases, that comprises an anti-caspase antibody, or fragment thereof, conjugated to a membrane transporter peptide, e.g., an MTS peptide. The antibody-peptide conjugates of the present invention are capable of causing internalization of the antibody or antibody fragment into cells.

In another aspect of the invention, a method of treating or preventing a disease comprises administering to a patient in need thereof a pharmacologically effective amount of a pharmaceutical composition comprising an anti-caspase antibody, or fragment thereof, conjugated to a membrane transporter peptide or fragment thereof. Specifically demonstrated are modified anti-caspase antibodies conjugated to a membrane transporter peptide that reduce chemically induced apoptosis. These results suggest such antibodies have therapeutic potential to inhibit apoptosis in a variety of diseases, such as Alzheimer's, Huntington's or Parkinson's.

The above and other objects of the invention will become readily apparent to those of skill in the relevant art from the following detailed description and figures, wherein only preferred embodiments of the invention are shown and described. As is readily recognized, the invention is capable of modifications within the skill of the relevant art without departing from the spirit and scope of the invention.

### BRIEF DESCRIPTION OF DRAWINGS

FIG. 1 shows the detection viability of MTS-anti-active caspase-3 antibody conjugate-treated Jurkat cells. 2.5.times.10.sup.5 Jurkat cells were seeded into 96-well culture plate. After incubation with 0.5 .mu.g MTS-antibody for 6, 12, 18 and 24 hour, aliquots were removed and viable cells were counted using dye exclusion (trypan blue).

FIG. 2 depicts detection of antibody internalization by sandwich ELISA. Sheep anti-rabbit antibody was coated onto an ELISA plate (400 ng/well). The cell homogenate and equal volume of the culture supernatant were added to a sheep anti-rabbit IgG-coated ELISA plate (Falcon, Oxnard, Calif.) and incubated for 2 h at room temperature. After washing, HRP-labeled gloat anti-rabbit light chain antibody was added, and antibody was visualized by adding o-phenylene-diamine. The ratio of internalized antibody versus culture antibody is plotted.

FIG. 3 depicts the extent of DNA fragmentation measured by cell death ELISA assay. MTS-conjugated or naked anti-caspase-3 antibody (2 .mu.g/ml) was added to 6-ml cultured Jurkat cells and pre-incubated for 1 h. The antibody was washed out by centrifugation, fresh medium was added containing actinomycin D (1 .mu.g/ml), and incubating for 4 h. 5 ml of the culture was collected for DNA fragmentation assessment by ladder electrophoresis; the rest for the ELISA assay. AD=actinomycin D; Naked Ab=caspase-3 antibody; MTS-Ab=MTS-conjugated anti caspase-3 antibody; Caspase-3 inhibitor-DEVD-fmk (100 .mu.M). *,p<0.01 comparing with Control; #, p<0.01 comparing with naked caspase-3 antibody.

FIG. 4 depicts caspase-3-like cleavage activity assay. An equal amount of protein of the total cell lysate was applied for the assay by using the ApoAlert Caspase-3 Fluorescent Assay Kit. *, p<0.01 comparing with Control; #, p<0.01 comparing with naked caspase-3 antibody.

### DETAILED DESCRIPTION OF THE INVENTION

The present invention describes a method for creating fusion proteins of an antibody and a peptide having a biological activity selected from the group consisting of immuno-stimulatory, membrane transport and homophilic activities.

In particular, the present invention provides a fusion protein comprising an antibody and a peptide having a biological activity selected from the group consisting of immuno-stimulatory, membrane transport and homophilic activities, wherein the peptide is located at a site in the antibody so that the incorporated peptide does not compromise the antigen recognition of the antibody. In the present invention, this is accomplished by a method comprising the steps of creating a fusion gene comprising a nucleic acid sequence encoding an antibody and a nucleic acid sequence encoding the peptide, wherein the nucleic acid sequence encoding the peptide is located inside the nucleic acid sequence encoding the antibody at a site wherein, when the fusion is expressed, the fusion protein that is created thereby includes the antibody plus the peptide, and the peptide is connected to the antibody at a site that does not interfere with antigen binding of the antibody, and expressing the fusion gene to create the fusion protein. In particular, the fusion protein may be created by providing a gene encoding an antibody, wherein the gene is mutated to contain a restriction site, wherein the restriction site is located away from any section of the gene that encodes an antigen-binding site of the antibody, inserting a DNA sequence encoding a peptide having a biological activity selected from the group consisting of immuno-stimulatory, membrane transport and homophilic activities into restriction site of the gene encoding the antibody to create a fusion gene, and wherein the DNA sequence encoding the peptide is inserted so that it is in-frame with the gene encoding the antibody, and expressing the fusion gene to create a fusion protein.

In a further embodiment of the present invention, the peptide having biological activity may be attached to the C-terminus of the antibody. In a further embodiment of the present invention, the peptide may be flanked by loop-forming or conformation-conferring sequences to enhance the biological activity of the peptide.

As used herein, the term “targeting, moiety” refers to any natural or synthesized protein molecule containing an antigen-binding site. The term includes a full-length immunoglobulin molecule or any functional fragment, such as a variable domain fragment of a full-length immunoglobulin molecule, CDR regions, ScFv, Fab, F(ab)′2, or engineered antibody mimics or single domain binding moieties. A particular targeting moiety is selected in accordance with the desired target, such as a cellular receptor on a membrane structure, e.g., a protein, glycoprotein, polysaccharide or carbohydrate. The targeting moiety can be selected to bind a cellular receptor on a normal cell or on a tumor cell.

Likewise, the peptide having biological activity is selected according to the desired function of the fusion protein, or, in other words, according to the desired result after the targeting moiety binds to a target such as a normal cell or a tumor cell. Possible biological activities that may be desired include immuno-stimulatory, membrane transport and homophilic activities.

The loop-forming or conformation-constraining sequences may be any amino acid sequences that, when placed on either side of the peptide having biological activity, restrain the conformational flexibility of the peptide. Examples include sequences containing amino acid residues such as cysteine pairs that can cross-link to form loops. A specific example of a conformation-constraining protein is thioredoxin. Examples of conformation-constraining or loop-forming moieties may be found, for example, in the following U.S. Pat. Nos. 6,242,163 and 6,004,746 to Brent, U.S. Pat. Nos. 6,258,550; 6,147,189; 6,111,069; 6,100,044; 6,084,066; 5,952,465; 5,948,887; and 5,928,896 to Brent et al, U.S. Pat. Nos. 6,200,759 and 5,925,523 to Dove et al., and in the following publications:
Fairlie D P; West M L; Wong A K “Towards protein surface mimetics.” Curr Med Chem 1998 February; 5 (1):29-62;
Valero M L; Camarero J A; Haack T; Mateu M G; Domingo E; Giralt E; Andreu D “Native-like cyclic peptide models of a viral antigenic site: finding a balance between rigidity and flexibility.” J Mol Recognit 2000 January-February; 13(1):5-13;
Gururaja T L; Narasimhamurthy S; Payan D G; “A novel artificial loop scaffold for the noncovalent constraint of peptides.” Chem Biol. 2000 July; 7(7):515-27;
Venkatesh N; im S H; Balass M; Fuchs S; Katchalski-Katzir E “Prevention of passively transferred experimental autoimmune myasthenia gravis by a phage library-derived cyclic peptide.” Proc Natl Acad Sci USA 2000 Jan. 18; 97(2):761-6;
Stott K; Blackburn J M; Butler P J; Perutz M “Incorporation of glutamine repeats makes protein oligomerize: implications for neurodegenerative diseases.” Proc Natl Acad Sci. USA 1995 Jul. 3;

All of the above are incorporated herein by reference.

The conformation-constraining sequences may also include sequences that form alpha helices or beta-pleated sheets. See, for example, the following publications incorporated herein by reference:
Lee K H; Benson D R; Kuczera K “Transitions from alpha to pi helix observed in molecular dynamics simulations of synthetic peptides.” Biochemistry 2000 Nov. 14; 39(45): 13737-47;
Dettin M; Roncon R; Simonetti M; Torinene S; Falcigno L; Paolillo L; Di Bello C “Synthesis, characterization and conformational analysis of gp120-derived synthetic peptides that specifically enhance HIV-1 infectivity.” J Pept Sci 1997 January-February; 3 (1):15-30;
Chavali G B; Nagpal S; Majumdar S S; Singh O; Salunke D M “Helix-loop-helix motif in GnRH associated peptide is critical for negative regulation of prolactin secretion.” J Mol. Biol. 1997 Oct. 10; 272(5):731-40; and
Miceli R; Myszka D; Mao L I; Sathe G; Chaiken I “The coiled coil stem loop miniprotein as a presentation scaffold.” Drug Des Discov., 1996 April; 13 (3-4): 95-105.

The Expression of 1′-fusion Proteins. Ig fusion proteins have the advantage of joining the antibody combining specificity and/or antibody effector functions with molecules contributing unique properties. The ability to produce this family of proteins was first demonstrated when c-myc was substituted for the Fc of the antibody molecule, (Neuberger M S, Williams G T and Fox R O., Nature, 125:604, 1984) but many examples now exist. Ab fusion proteins can be achieved in several different ways. In one approach non-Ig sequences are substituted for the variable region; the molecule replacing the V region provides specificity of targeting with the antibody contributing properties such as effector functions and improved pharmacokinetics. Examples include IL-2 and CD4. Alternatively, non-Ig sequences can be substituted for or attached to the constant region. The resulting molecules retain the binding specificity of the original antibody but gain characteristics from the attached protein. Depending on the position of the substitution, different antibody-related effector functions and biologic properties will be retained. See, for example, Antibody Engineering, 2nd Edition. ed.: Carl A. K. Borrebaeck, Oxford University Press, 1995)

Vectors for the Construction of IgG Fusion Proteins. A series of vectors has now been produced that permits the fusion of proteins at different positions within an antibody molecule, thereby facilitating the construction of fusion proteins with different properties. Using these vectors it is possible to produce a family of fusion proteins with molecules of differing molecular weight, valence, and having different subsets of the functional properties of the antibody molecule.

As a specific example of how to facilitate the construction of fused genes, site-directed mutagenesis was used to generate unique restriction enzyme sites in the human IgG3 heavy chain gene. In this particular example, restriction sites were generated at the 3′ end of the CH1 exon, immediately after the hinge at the 5′ end of the CH2 exon, and at the 3′ end of the CH3 exon. The restriction sites thus produced were SnaB I at the end of CH1 by replacing TtgGTg, with TacGTa, Pvu II at the beginning of CH₂ by replacing CAcCTG with CAgCTG, and Ssp I at the end of CH3 replacing AATgag with AATatt. These manipulations provided a unique blunt-end cloning site at these positions. In all cases the restriction site was positioned so that after cleavage the Ig would contribute the first base of the codon. Human IgG3 with an extended hinge region of 62 amino acids was chosen for use as the immunoglobulin; when present this hinge should provide spacing and flexibility, thereby facilitating simultaneous antigen and receptor binding. An EcoR I site was also introduced at 3′ of the IgCG3 gene to provide a 3′ cloning site and polyA addition signal. Although initially designed for use with growth factors, these restrictions sites can be used to position any novel sequence at defined positions in the antibody. Also, using these cloning cassettes the variable region can easily be changed. Similar techniques may be used to generate suitable restriction sites in other antibody genes.

Production of a Fusion Gene. As a first step in the production of a fusion protein, a blunt-end restriction site must be introduced at the desired position into the 5′ end of the gene to be fused. In order to maintain the correct reading frame, the site must be positioned so that after cleavage it will contribute two bases to the codon. If the objective is to make a fusion protein with the complete molecule, the restriction site is usually introduced at the position of any post-translational processing, such as after the leader sequence. Alternatively, if the objective is to use only a portion of the protein, the blunt-end site can be introduced at any position within the gene, but attention must always be paid to maintaining the correct reading frame. Additionally, if there is carboxyl-terminal post-translational processing of the fused protein, it is frequently desirable to introduce a stop-codon at this processing site.

A major concern when producing fusion proteins is maintaining the biologic activities of all of the components. The production of fusion proteins with antibodies is facilitated by the domain structure of the antibody, and all of the cloning sites have been positioned immediately following an intact domain. In this configuration the correct folding of the immunoglobulin should be assured. The folding of the attached protein depends on its structure and where it is fused. Whenever structural information is available, it is desirable to produce the fusion at a position that will maintain the structural integrity of the attached protein.

To produce quantities of protein sufficient for functional analysis, it is desirable to have the protein secreted into the medium. While in the examples reported to date, assembled fusion proteins have been assembled and secreted, this remains a concern when designing additional fusion proteins.

The method to design a fusion gene that contains a biologically activity peptide as part of the heavy or light chain gene can use established antibody engineering protocols (Antibody Engineering 2nd Edition. ed.: Carl A. K. Borrebaeck, Oxford University Press 1995. Chapter 9, pages 267-293). The peptide can fused either to N-terminal residues or the C-terminal residues of H or L chains. The expression of such fused genes is typically done in mammalian cell lines, although other expression systems, such as, for example, bacteria or yeast expression systems, may be used.

The peptide of the invention has a biological activity selected from the group consisting of immuno-stimulatory, membrane transport and homophilic activities. Examples include immuno-stimulatory or immuno-regulatory activity. The peptide may, for example, be a hormone, ligand for cytokines or a binding site derived from natural ligands for cellular receptors. In a preferred embodiment, the peptide is derived from C3d region 1217-1232 and ranges from about 10 to about 16 mer. In an alternative embodiment, the peptide is a 16 mer peptide derived from the C3d region 1217-1232.

The peptide may be bound to an antibody that is a full-length immunoglobulin molecule or a variable domain fragment of an antibody. As used herein, the term “antibody” refers generally to a heavy or light chain immunoglobulin molecule or any function combination or fragment thereof containing an antigen-binding site. The antibody is preferably specific for a cellular receptor, on a membrane structure such as a protein, glycoprotein, polysaccharide or carbohydrate, and on a normal cell or on tumor cells.

The use of peptides derived from the ligand site of C3d as an immunostimulatory component incorporated into antibodies has an unexpected utility as a molecular adjuvant. C3d has been used as molecular adjuvant as part of a complete fusion protein with hen egg lysozyme (HEL) by D. Fearon, et al., (Dempsey, P. W., Allison, M. E. D., Akkaraju, S., Goodnow, C. C. and Fearon, D. T., Science, 271:348, 1996). These authors have shown that a HEL-C3d fusion protein is up to 10,000 fold more immunogenic than free HEL (see International Patent Publication, WO96/17625).

Similar increases in immunogenicity have been observed with chemical cross-linked idiotype vaccines using a peptide derived from the C3d fragment in our recent animal studies (see examples below). It is believed that attaching C3d peptides to idiotype and anti-idiotype vaccines enhances the immunogenicity of these vaccines and substitutes for the need of attaching carrier molecules such as KLH in combination with strong adjuvants, such as Freund's adjuvant, which is not permitted by the FDA for use with humans.

In an alternative embodiment, the peptide may be derived from a human or non-human C3d region homologous to the human C3d residues at position 1217-1232 and ranges from about 10 to about 16 mer. Other applications of affinity cross-linking biologically active peptides to antibody vaccines include active peptides derived from cytokines. For example, a nonapeptide from the IL1-beta cytokine has been described (Antoni, et al., J Immunol, 137:3201-04, 1986) which has immunostimulatory properties without inducing undesired side effects. Other examples of active peptides which can be inserted into antibodies in accordance with the invention include signal peptides, and peptides from the self-binding locus of antibodies.

A variety of peptides are known having biological activities as hormones, ligands for cytokines or binding sites derived from natural ligands for cellular receptors.

The following Examples 1-3, while relating to C3d/antibody complexes that are created by affinity cross-linking, are provided to show the effects on the immune response provided by C3d peptides linked to antibodies.

### Example 1

### Enhancement of an Anti-Idiotype Vaccine

3H1 is a murine anti-idiotype antibody (Bhattacharya-Chatterjee, et al. J Immunol., 145:2758-65, 1990) which mimics the carcino-embryonic antigen (CEA). 3H1 induces in animals anti-CEA antibodies when used as KLH-conjugated vaccine in complete Freund's adjuvant. 3H1 has also been tested in a clinical phase I study where it induces antibodies which bind to CEA in approximately half of treated cancer patients. However no clinical response was observed in this study (Foon, et al., J Clin. Invst., 96:334-342, 1995) due, in part, to low immunogenicity.

3H1 mAb was affinity cross-linked with a 13-mer peptide (SEQ ID NO.:1) derived from the C3d region 1217-1232. The amino acid sequence was derived from of the Cd3 peptide and has the following sequence: KNRWEDPGKQLYNVEA (SEQ ID NO. 1)

BALB/c mice were immunized twice with 25 μg of C3d-3H1 in phosphate-saline solution intramuscular. 7 days after the last immunization mice were bled and sera were tested for binding to 8019 (Ab1 idiotype) and to the CEA expressing tumor line LS174T. As determined by FACS, sera from C3d-3H1 immune mice bind to LS174T tumor cells, while a control serum (normal mouse serum) showed only background fluorescence. Sera from mice immunized with C3d-3H1 were used in FACS of LS174T cells in a sandwich assay developed with FITC-conjugated goat anti-mouse IgG. Control was a normal mouse serum. Cell numbers analyzed were plotted against relative fluorescence intensity on log 10 scale.

### Example 2

Furthermore, sera from mice immunized three times with either 3H1 (25 microgram in saline) or 3H1-C3d-peptide (affinity cross-linked, 25 microgram in saline) were also tested for Ab3 response. Mice were bled and sera were tested for binding to F(ab) of 3H1 in ELISA. Upon determining the binding of dilutions of mouse sera to 3H1 F(ab), it was found that while naked 3H1 does not induce Ab3 antibodies, 3H1-peptide does showing that the affinity-cross-linked 3H1 enhanced immunogenicity.

Other C3d peptides which may be used in the practice of the present invention include those reviewed in Lambris et al, “Phylogeny of the third component of complement, C3” in Erfi, A ed. New Aspects of Complement structure and function, Austin, R. D. Landes Co., 1994 p. 15-34, incorporated herein by reference in its entirety.

### Example 3

Enhancement of an Mouse Tumor Idiotype Vaccine (38C13)

38C13 is the idiotype expressed by the 38C13 B-lymphoma tumor cell line. The Levy group has developed this idiotype tumor vaccine model and has shown that pre-immunization with KLH-conjugated 38C13 Id can protect against challenge with 38C13 tumor cells in mice (Kaminski, M. S., Kitamura, K., Maloney, D. G. and Levy, R., J Immunol., 138:1289, 1987). Levy and colleagues (Tao, M-H. and Levy, R., Nature, 362:755-758, 1993) have also reported on the induction of tumor protection using a fusion protein (CSF-38C13), generated from a chimeric gene and expressed in mammalian cell culture fermentation. 38C13 Id proteins were affinity cross-linked with a 16-mer azido-peptide derived from the C3D region 1217-1232.

Ten mice were immunized with 50 ug of C3d-38C13 conjugate in phosphate-saline solution intra-peritoneally three times. After the third vaccination mice were challenge with 38C13 tumor cells. Control groups included mice vaccinated with 38C13-KLH in QS-21 (adjuvant), considered the “gold standard” in this tumor model, and mice injected with QS-21 alone. Seven out of ten mice vaccinated with the C3d-38C13 conjugate survived by day 35 after tumor challenge, as did mice vaccinated with the KLH-38C13 in QS-21. All control mice injected only with QS-21 died by day 22.

C3H mice were immunized three times with either 38C13-KLH in QS-21 or with 38C13-C3d peptide without QS-21 (50 μg i.p.) Control mice were only injected with QS-21. Immunized and control mice were than challenged with 38C13 tumor cells and survival was monitored.

Results described in Examples 1-3 show that affinity-cross-linking of an immuno-stimulatory peptide to tumor anti-idiotype and idiotype vaccine antibodies can significantly enhance the immune response to the tumor-and protect against tumor challenge. The vaccination protocol with the C3d-cross-linked vaccine did not include any adjuvant, such as Freund's adjuvant, or KLH conjugation, both of which are not permissible by the FDA for human use. Some of the procedures used in the above examples are known; the active binding peptide of C3d (complement fragment) has been described by Lambris, et al., (PNAS, 82:4235-39, 1985) and is incorporated herein by reference in its entirety.

The following additional examples are provided to demonstrate the general technique of creating fusion proteins and to illustrate particular peptide having a biological activity selected from the group consisting of immuno-stimulatory, membrane transport and homophilic activities.

### Example 4

Fusion non-Ig Protein Containing a Membrane Transferring Peptide (MTS-peptide)

See, e.g., Rojas, M, Donahue, J P, Tan, T. and Lin, Y-Z. Nature Biotech., 16: 370, “Construction of the glutathion S-transferase-MTS peptide (GST-MTS) expression plasmids,” 1998.

Two different GST-MTS expression plasmids were constructed so that, depending on the biological application, a target protein or protein domain could be produced with the hydrophobic MTS as either an amino-terminal or a carboxyl-terminal extension. For the construction of plasmids pGEX-3X-MTS I and pGEX3X-MTS2, the following complementary oligonucleotides were synthesized:

1

(SEQ ID NO. 2)

MTSI:
GATCGCAGCCGTTCTTCTCCCTGTTCTTCTTGCCGC-ACCCGG-C
GTCGGCAAGAAGAGGGACAAGAAGAACGGCGTGGGCCCTAG

(SEQ ID NO. 3)

MTS2:
GATCCCCGCAGCCGTTCTTCTCCCTGTTCTTCTTGCCGCACCC-T AGC-
GGGCGTCGGCAAGAAGAGGGACAAGAAGAACGGCGTGGGA TTCGCTAG

After annealing, the double-stranded MTS I and MTS2 oligonucleotides were ligated in BamHI digested pGEX-3X (Smith, D. B. and Johnson, K. S., “Single-step purification of polypeptides expressed in Escherichia coli as fusions with glutathione S-transferase,” Gene, 67:31. 1988.). DNA sequence analysis confirmed that in each plasmid the MTS coding sequence was correct and in-frame with the GST coding sequence.

Construction of GST-Grb2SH2, GST-Grb2SH2-MTS, and GST-Stat1SH2-MTS Expression Plasmids.

A DNA fragment encoding the human Grb2 SH2 domain (amino acid residues 54-164) (Lowenstein, E. J., Daly, R. J., Batzer, A. G., U, W., Margolis, B., Lammers, R et al., “The SH2 and SH3 domain-containing protein Grb2 links receptor Lyrosine kinases to ras signalings,” Cell, 70:431, 1992) or the human Stat1 SH2 domain (residues 567-716) (Schindler, C., Fu, X.-Y, Impnota, T., Aebersold, R., and Darnell, J. E. Jr., Proc. Natl. Acad. Sci. USA 89:7836, 1992) was synthesized from a Grb2 cDNA clone or a Stat1 cDNA clone by PCR. The primers used for PCR, each containing BamHI sites at their 5′ ends, were as follows:

2

(SEQ ID NO. 4)

Grb2 SH2:
5′-CCGGATCCCCGAAATGAAACCACATCCGTGGTTTTTTGGC
and

(SEQ ID NO. 5)

5′-CCGGATCCCGAGGGCCTGGACGTATGTCGGCTGCTGTGG.

(SEQ ID NO. 6)

Stat1 SH2:
5′-CCGGATCCCCAAACACCTGCTCCCTCTCTGGAATGATGGG
and

(SEQ ID NO. 7)

5′-CCGGATCC-CTCTAGAGGGTGAACTTCAGACACAGAAAT.

The PCR products were digested with BamHI and ligated in BamHI-digested pGEX-3X or pGEX-3XMTS2. DNA sequence analysis of the vector/insert junctions confirmed that the GST-Grb2SH2, GST-Grb2SH2-MTS, and GST-Stat1SH2-MTS translational reading frames were maintained in each expression plasmid.

Expression of MTS Fusion Protein

Expression and purification of GST fusion proteins. E. coli strain DHS or containing the appropriate expression plasmid 74 as grown in LB broth containing 100 μg/ml ampicillin at 37° C. GST fusion protein expression was induced by the addition of isopropyl, B-D-thiogalactoside (0.5 mM final concentration), and incubation at 37° C. was continued for 2-3 hours. GST fusion proteins were purified from bacterial cell lysates by glutathione-agarose affinity chromatography. (Smith, D. B. and Johnson, K. S. Gene, 67:31, 1988) except that after sonication, cell lysates were cleared by centrifugation at 2000×g for 5 minutes prior to mixing with glutathione-agarose beads. Protein preparations were concentrated by ultrafiltration using a PMIO membrane (Amicon, Beverly, Mass.) and stored at 4° C. for immediate use or −70° C. for prolonged storage. Protein concentrations were determined spectrophotometrically at 280 nm. Immediately prior to their use in biological assays, protein concentrations were verified by SDS-PAGE using Coomassie brilliant blue staining intensity compared with wild-type GST of known concentration. To confirm the amino acid content of the MTS in GST-MTS proteins, the MTS peptide was cleaved from glutathione-agarose bound GST-MTSI with protease factor Xa essentially as described (Smith, D. B. and Johnson, K. S., Gene 67:31, 1988). The released MTS-containing peptide was purified by C, reverse-phase HPLC and characterized by mass spectrometry analysis as described (Smith, D. B. and Johnson, K. S., Gene 67:31, 1988). The released MTS-containing peptide was purified by C₁₈ reverse-phase HPLC and characterized by mass spectrometry as described (Lin, Y-Z., Yao, S., Veach, R. A., Torgerson, T. R., and Hawiger, J., J. Biol. Chem. 270:14255, 1995).

### Example 5

C3d-HEL Fusion Protein (Dempsey et al., Science, 271: 348, 1996)

The complimentary DNA encoding HEL, C3d (H. Domdey et al., Pro. Natl Acad Sci USA, 79: 7619, 1982) doq pre-pro-insulin signal sequence (M. E. Taylor and K. Drickamer, Biochem. J, 274, 575, 1991), and the (G4S)₂ linker were amplified by polymerase chain reaction. The epitope tag and stop codon were coded for by oligonucleotide linkers. Fusion protein cassetes were assembled in tandem: doq pre-pro-insulin signal sequence, HEL, and one to three copies of C3d linked by (G4S)₂ in pSG5 (Stratagene Cloning Systems, La Jolla, Calif.). The HEL-C3d3 cassette was subcloned into the A71d vector. The plasmids pSG.HEL, pSG.HEL.C3d, and pSG.HEL.C3d2 were co-transfected with pSV2-neo into L cells and A71d. HEL.C3d3 was transiently expressed in COS cells. Recombinant proteins were purified by affinity chromatography on YL 1/2 antibody (H. Skinner et al., J. Biol. Chem., 66: 14163, 1991) and fractionation on Sephacryl S-200 (Pharmacia).

Fusion tails are useful at the lab scale and have potential for enhancing recovery using economical recovery methods that are easily scaled up for industrial downstream processing. Fusion tails can be used to promote secretion of target proteins and can also provide useful assay tags based on enzymatic activity or antibody binding. Many fusion tails do not interfere with the biological activity of the target protein and in some cases have been shown to stabilize it. Nevertheless, for the purification of authentic proteins a site for specific cleavage is often included, allowing removal of the tail after recovery.

Fusion Tails for the Recovery and Purification of Recombinant Proteins.

(See, e.g., Ford C., Suominen I., Glatz C., Protein Expr. Purif 2-3: 95-107, 1991). The fusion protein of the present invention may also include a fusion tail such as has been developed to promote efficient recovery and purification of recombinant proteins from crude cell extracts or culture media. In these systems, a target protein is genetically engineered to contain a C- or N-terminal polypeptide tail, which provides the biochemical basis for specificity in recovery and purification. Tails with a variety of characteristics have been used:

(1) entire enzymes with affinity for immobilized substrates or inhibitors;

(2) peptide-binding proteins with affinity to immunoglobulin G or albumin;

(3) carbohydrate-binding, proteins or domains;

(4) a biotin-binding domain for in vivo biotinylation promoting affinity of the fusion protein to avidin or streptavidin;

(5) anti genic epitopes with affinity-to immobilized monoclonal antibodies;

(6) poly(His) residues for recovery by immobilized metal affinity chromatography; and

(7) other poly(amino acid)s, with binding specificity based on properties of the amino acid side chain.

Fusion tails are useful at the lab scale and have potential for enhancing recovery using economical recovery methods that are easily scaled up for industrial downstream processing. Fusion tails can be used to promote secretion of target proteins and can also provide useful assay tags based on enzymatic activity or antibody binding. Many fusion tails do not interfere with the biological activity of the target protein and in some cases have been shown to stabilize it. Nevertheless, for the purification of authentic proteins, a site for specific cleavage is often included, allowing removal of the tail after recovery.

The present invention describes the generation of an antibody-peptide fusion protein that enhances the biological and immunological activity of the antibody without changing the antibody specificity for the corresponding antigen. The genetically engineered fusion protein mimics the chemically engineered chimeric antibodies described in U.S. Pat. No. 6,238,667. Specifically, the present invention provides the generation of antibody fusion proteins containing the complete or partial autophilic 24-mer peptide, the membrane transport peptide (MTS) or the C3d peptide, all described above.

The invention also provides a composition and a pharmaceutical composition comprising a fusion protein made up of (1) an antibody and (2) a peptide having a biological activity selected from the group consisting of immuno-stimulatory, membrane transport and homophilic activities wherein the peptide is connected by peptide bonds to the antibody at a site that does not interfere with antigen binding of the antibody.

Any antibody may be used in the peptide/antibody complex of the invention. Preferred antibodies are anti-idiotype antibodies. For example, anti-idiotype antibody 3H1 may be used (see “Anti-idiotype Antibody Vaccine (3H1) that Mimics the Carcinoembryonic Antigen (CEA) as an Adjuvant Treatment”, Foon, et al., Cancer Weekly, Jun. 24, 1996). Other anti-idiotype antibodies which may be used in the present invention include, for example, anti-idiotype antibody to chlamydia glycolipid exoantigen (U.S. Pat. No. 5,656,271; anti-idiotype antibody 1A7 for the treatment of melanoma and small cell carcinoma (U.S. Pat. No. 5,612,030); anti-idiotype antibody MK2-23 anti-melanoma antibody (U.S. Pat. No. 5,493,009); anti-idiotypic gonococcal antibody (U.S. Pat. No. 5,476,784) Pseudomonas aeruginosa anti-idiotype antibody (U.S. Pat. No. 5,233,024); antibody against surface Ig of human B cell tumor (U.S. Pat. No. 4,513,088); and monoclonal antibody BR96 (U.S. Pat. No. 5,491,088). Any restrictions on peptide length are those practical limitations associated with peptide synthesis and not restrictions associated with practice of the method of the invention.

Additionally, self-binding peptides such as those disclosed in (Kang, C. Y. Brunck, T. K., Kiever-Emmons, T., Blalick, J. E. and Kohler, H., “Inhibition of self-binding proteins (auto-antibodies) by a VH-derived peptide, Science, 240: 1034-1036, 1988, which is incorporated herein by reference in its entirety) may be used in the method of the present invention.

Additionally, signal peptides such as those disclosed in Rojas, et al., “Genetic Engineering of proteins with cell membrane permeability”, Nature Biotechnology, 16: 370-375 (1988) and Calbiochem Signal Transduction Catalogue 1997/98, incorporated herein by reference in their entireties, may be used in the method of the invention.

The peptide may be designed to have inverse hydropathic character and exhibits mutual affinity and homophilic (self) binding within the peptide, in accordance with the disclosure of U.S. Pat. No. 5,523,208 (incorporated herein by reference in its entirety).

The present invention contemplates novel compounds and methods for regulating cellular functions, either in normal or infected cells. Such compounds comprise an antibody, or fragment thereof which is capable of being internalized within the cell through the cell penetrating action of a peptide conjugated thereto. Such peptides are referred to herein as “membrane transporter peptides,” and the like. Known membrane transporter peptides, or their active fragments, can be employed as the attached peptide. Such antibodies, or fragments thereof, are immunospecific for such protein targets as: (a) signaling proteins internal the cell, such as caspases, kinases, and phosphatases, (b) immature virion proteins prior to intracellular assembly, (c) cell-surface or intracellular tumor antigens, (d) nuclear or nucleolar proteins that are involved in regulation of DNA synthesis and gene expression, or (e) cytoskeletal proteins that participate in cell proliferation or cytostasis. Either polyclonal or monoclonal antibodies can be used. Such antibodies or their fragments preferably bind to their bind to their determinants with an affinity of 10.sup.-9M or greater.

A particularly preferred compound of the invention is one that comprises an anti-caspase antibody conjugated to a membrane transporter protein, or peptide fragment thereof. A preferred membrane transporter fragment is a membrane translocation sequence (MTS) peptide. Particularly preferred membrane transporter peptides include the following:

(1) KGEGAAVLLPVLLAAPG (SEQ ID NO: 8), derived from Kaposi fibroblast growth factor [K-FGF] (Rojas et al., Nature Biotechnology, 16: 370-375 (1998)).

(2) AAVLLPVLLAAP (SEQ ID NO: 9), a truncated version of above peptide, see, Lin et al., J. Biol. Chem., 271: 5305 (1996).

(3) RQIKIWFQNRRMKWKK (SEQ ID NO: 10), “penetratin” derived from the homeodomain of Antennapedia (Ant) (see, Lindberg, M. et al., Eur. J. Biochem., 270(14): 3055-3063 (2003)).

(4) RRMKWKK (SEQ ID NO: 11), the C-terminal sequence of penetratin, see, e.g., Fischei, P. et al. J Peptide Res., 55(2): 163-172 (2000).

(5) TAT peptides, e.g., aa 47-57 and 48-60 derived from HIV-1 TAT (see, e.g., Schwarze, S., et al., Trends Pharmacol. Sci., 21: 45, 2000; Li Y., et al. Biochem.Biophys. Res. Commun. 298(3): 439-449, 2002; Hallbrink M., et al. Biochim. Biophys.Acta, 1515(2): 101-09, 2001).

(6) Herpes virus protein VP22 (Elliot, G., et al., Cell, 88: 223 (1997)).

(7) GWTLNSAGYLLGKINLKALAALAKKIL (SEQ ID NO: 12), “transportan,” a 27-mer peptide (see, Pooga, M. et al., FASEB J, 12: 67 (1998); Lindberg, M. et al. Biochem., 40: 3141-3149, 2001).

(8) AGYLLGKINLKALAALAKKIL (SEQ ID NO: 13), N-terminal six residue deletion of transportan (see, Soomets, U. et al., Biochim.Biophys.Acta, 1467:165-176, 2000).

(9) Lys-Leu-Ala-Leu (KLAL) (SEQ ID NO: 14), also referred to as MAP (see, Halibrink M., et al. Biochim. Biophys.Acta, 1515(2): 101-109, 2001).

Also contemplated is a pharmaceutical composition effective in inhibiting apoptosis that comprises an anti-caspase antibody conjugated to a membrane transporter protein or fragment thereof, as discussed herein. Such fusion proteins and methods of generating them are disclosed in U.S. Ser. No. 09/865,281 (Kohler et al.), incorporated herein by reference.

A preferred immunoconjugate of the present invention comprises a secondary antibody conjugated to an MTS sequence through one of several types of linkages including through the nucleotide or tryptophan sites of the antibody or through the N-linked carbohydrate of the antibody. A “secondary antibody,” as used herein, refers to an antibody, or fragment thereof, that binds specifically and with high affinity to a primary antibody. The secondary antibodies useful for the present invention include polyclonal or monoclonal antiglobulins to murine or human IgC or secondary antibodies targeted to novel and/or installed sequences such as the T15 sequence (Kang, C Y, Brunck, T K, Kieber-Emmons, T, et. al. “Inhibition of self-binding antibodies (autobodies) by a VH-derived peptide,” Science, 240:1034-6, 1988), which imparts autophilicity to an antibody.

Delivery is accomplished by pre-administering or pre-injecting a monoclonal antibody or immunoconjugate, targeted to a cell-surface antigen, allowing sufficient time for binding to the target and clearance from the tissues, and following with administration of a secondary antibody covalently linked to a MTS peptide. The primary antibody can be conjugated to an inhibitor, such as a toxin, drug, enzyme or isotope, thereby enhancing delivery of an inhibitory molecule into the cell. The secondary antibody conjugated to MTS peptide recognizes and binds to the primary antibody, and is internalized into the cell through the MTS peptide activity. In this way, the primary immunoconjugate is brought into the cell where its inhibitory action is enhanced.

Such secondary conjugates can also be used to assess the utility of monoclonal antibodies to intracellular targets by admixing primary and secondary antibodies conjugated to MTS, then exposing cells and testing for inhibition of cellular activities targeted with the primary antibody. In this rapid screen, many antibodies to intracellular targets can be screened for utility as antagonists or agonists. Those with activity can then be directly conjugated to a membrane transporter peptide, such as MTS, for in vivo use.

A preferred embodiment of the current invention utilizes MTS peptides conjugated to a tryptophan or nucleotide binding site of a secondary antibody and a primary antibody, conjugated to a toxin, drug or isotope attached through a sulfhydryl, epsilon amino acid or carbohydrate residues via chemical or peptide linkers or chelates.

The present invention relates generally to the in vivo delivery of antibody conjugates into the interior of cells. Such antibodies can be potentially neutralizing, anti-viral antibodies, anti-regulatory protein antibodies, or anti-tumor antibodies. For example, delivery can be accomplished by administering to a living organism an antibody conjugate comprising a MTS peptide, and an antibody directed at determinants on a virus or other intracellular pathogen that are best expressed on immature virus or pathogen. Such conjugates have an increased opportunity for binding with high affinity, disrupting virus assembly and neutralizing virus before it has a chance to mature and infect other cells.

Thus, the current invention provides antiviral (anti-HIV) therapeutics as an example of a broader class of antibody therapeutics. The antibodies preferred in the current invention have the following preferred properties:

(1) They bind to antigens primarily expressed intracellularly. This includes tumor associated antigens (TAA) and viral glycoproteins. The former, includes TAA such as CEA. A particular determinant may be primarily associated with intracellular forms of the protein whereas others may be primarily expressed on the surface. Prior to this invention, most useful therapeutic antibodies have been selected for reactivity to cell surface molecules; with the ability to target intracellular antigens, selection criteria would include primary reactivity with intracellular antigen.

(2) Intracellular targets include viral glycoproteins. For instance, most monoclonal antibodies have been raised to virus propagated in cells for many passages rather than virus propagated in cells for only a few passages; as a result most monoclonals to viruses react better to laboratory strains of virus rather than fresh isolates. The proposed explanation for this difference in binding is that most antibodies, as with those to HIV, react to determinants that are cryptic and partially occluded on viral glycoproteins from low passaged virus (and presumably newly synthesized virus) because of higher glycosylation and folding of viral glycoproteins. This would mean that most antibodies should bind better to immature virions or incomplete virions that have under-glycosylated or incompletely glycosylated glycoproteins and/or ones that are not fully assembled. Thus, antibodies not considered useful for therapy because of limited reactivity with native virus, would, with access to intracellular, immature forms, be useful for targeting.

(3) They bind to a linear sequence of amino acids on TAA or viral glycoproteins rather than a conformation-dependent sequence. Such an antibody is more likely to bind to intracellular antigens, early in synthesis and maturation; this would include immature virions or non-assembled, glycoprotein precursors, present within cells.

(4) The antibodies should bind with an affinity of 10.sup.-9M or greater to their determinants.

It is now shown herein, by way of specific Examples, that a MTS-transport-peptide modified monoclonal anti-caspase-3 antibody reduces actinomycin D-induced apoptosis and cleavage of spectrin in living cells. These results suggest that such antibodies have a therapeutic potential to inhibit apoptosis in a variety of diseases.

### Example 6

### Cell Line and Antibodies

Human Jurkat T cell lymphomas were grown in RPMI 1640 supplemented with 10% fetal bovine serum and antibiotic (penicillin, streptomycin and amphetericin). Rabbit polyclonal anti-active caspase-3 antibody and anti-cleaved fodrin, i.e., alpha II spectrin, were purchased from Cell Signaling, Inc. (Beverly, Mass.). Rabbit monoclonal anti-active caspase-3 antibody was purchased from BD PharMingen (San Diego, Calif.). Rabbit anti-spectrin antibody was purchased from Cell Signaling (Beverly, Mass.). Mouse monoclonal antibody 3H1 (anti-CEA) was purified from cell-culture supernatant by protein G affinity chromatography. Anti-mouse and anti-rabbit HRP-conjugated secondary antibodies were purchased from Santa Cruz Biotechnologies, Inc. ApoAlert Caspase-3 Fluorescent Assay kit was purchased from Clontech Laboratories, (Palo Alto, Calif.). The Cell Death Detection ELISA was purchased from Roche Applied Science (Indianapolis, Ind.). Caspace inhibitors were purchased from Enzyme Systems Products (Livermore, Calif.).

### Example 7

### Synthesis of Antibody-Peptide Conjugate

MTS peptide (KCEGAAVLLPVLLAAPG) is a signal peptide-based membrane translocation sequence (1), and was synthesized by Genemed Synthesis (San Francisco, Calif.). Antibodies were dialyzed against PBS (pH6.0) buffer, oxidized by adding 1/10 volume of 200 mmol/L NaIO₄ and incubating at 4° C. for 30 min in the dark. The oxidation was stopped by adding glycerol to 30 mM and the sample was dialyzed at 4° C. for 30 min against PBS (pH6.0) buffer. 50 times more in molecules of MTS peptide was used to couple the antibodies by incubation at 37° C. for 1 h, then the antibody-peptide was dialyzed against PBS (pH 7.4).

### Example 8

### Effect of MTS-Conjugated Anti-Active Caspase-3 Antibody on Cell Growth

2.5×10⁵ Jurkat cells were seeded into 96-well culture plate. After incubation with 0.5 μg MTS-antibody conjugates for 6, 12, 18 and 24 hour, aliquots were removed and viable cells were counted using dye exclusion (trypan blue).

### Example 9

### Study of Antibody Internalization by ELISA

Jurkat cells, Crown in 1-ml medium, were incubated with 2 μg of naked or MTS-antibody conjugates for 0, 1, 3, 6, 19 and 18 h in 6-well culture plate (Costai, Cambridge, Mass.). The cells were spun down, the culture supernatant was transferred to a new tube and the cell pellet was washed twice with PBS (pH 7.4) before being homogenized by Pellet Pestle Motor (Kontes, Vineland, N.J.) for 30 sec. All the cell homogenate and equal volume (10 μl) of the culture supernatant were added to sheep anti-rabbit IgG coated ELISA plate (Falcon, Oxnard, Calif.) and incubated for 2 h at room temperature. After washing, HRP-labeled goat anti-rabbit light chain antibody was added, antibody was visualized using o-phenylenediamine.

### Example 10

### DNA Fragmentation

Jurkat cells were pre-treated with antibodies or caspase-3 inhibitor (DEVD-fmk) for 1 h, centrifuged, and incubated with fresh medium containing actinomycin D (1 .mu.g/ml) for 4 h. After treatment, Jurkat cells were collected and washed with PBS (pH 7.4), then suspended in 700 μl of BL buffer (10 mM Tris-HCl, pH 8.0, 1 mM EDTA, 0.2% Triton X-100), and incubated for 15 min at room temperature. Crude DNA preparations were extracted with phenol:chloroform:isoamyl alcohol (25:24:1) twice and precipitated for 24 h at −20° C. with 0.1 volume of 5 M NaCl and 1 volume of isopropanol. The collected DNA was dissolved in TE buffer (10 mM Tris, pH 8.0 with 1 mM EDTA). The same amount of DNA was resolved by electrophoresis on a 1.5% agarose gel and visualized by UV fluorescence after staining with ethidium bromide. DNA fragmentation was also detected by cell death detection ELISA (Roche, Indianapolis, Ind.), which was performed according to the manufacturer's instructions with minor modification: JB6 cells were crown in p100 plates, after treatment, cells were collected and 25 μl of the whole cell lysate were applied to each sample well.

### Example 11

### Preparation of Total Cell Lysate

Jurkat cells were treated the same way as in the previous section. After treatment, Jurkat cells were collected and washed with PBS (pH 7.4) twice, then were suspended in 300 μl of CHAPS buffer (50 mM PIPES, pH 6.5, 2 mM EDTA, 0.1% CHAPS). The samples were sonicated for 10 sec and centrifuged at 14,000 rpm for 15 min at 4° C. The supernatant was transferred to a new tube and referred to as “total cell lysate.”

### Example 12

### Caspase-3-Like Cleavage Activity Assay

Jurkat cells were treated the same way as in the previous section. Using equal protein concentrations of the total cell lysate and ApoAlert Caspase-3 Fluorescent Assay Kit, the caspase-3 activity was analyzed according to the manufacturer's instructions. Fluorescence was measured with a Spectra MAX GEMINI Reader (Molecular Devices, Sunnyvale, Calif.).

### Example 13

### Western Blot Analysis

Jurkat cells were treated the same way as in the previous section. 10 μg of the total cell lysate was separated on a 10% SDS-PAGE gel to detect immunoreactive protein against cleaved spectrin (1:1000 dilution). Ponceau staining was used to monitor the uniformity of transfer of protein onto the nitrocellulose membrane. The membrane was washed with distilled water to remove excess stain and blocked in Blotto (5% milk, 10 mm Tris-HCl [pH 8.0], 150 mM NaCl and 0.05% Tween 20) for 2 h at room temperature. Before adding the secondary antibody, the membrane was washed twice with TBST (10 mM Tris-HCl with 150 mM NaCl and 0.05% Tween 20), and then the membrane was incubated with horseradish peroxidase-conjugated secondary antibodies (Santa Cruz Biotechnology) at a 1:4000 dilution. The final washing steps included three times (5 min each) with TBST and two times (5 min each) with TBS (10 mM Tris-HCl with 150 mM NaCl). The antibody bands were visualized by the enhanced chemiluminescence detection system (ECL, Amersham Pharmacia Biotech, Piscataway, N.J.).

Results

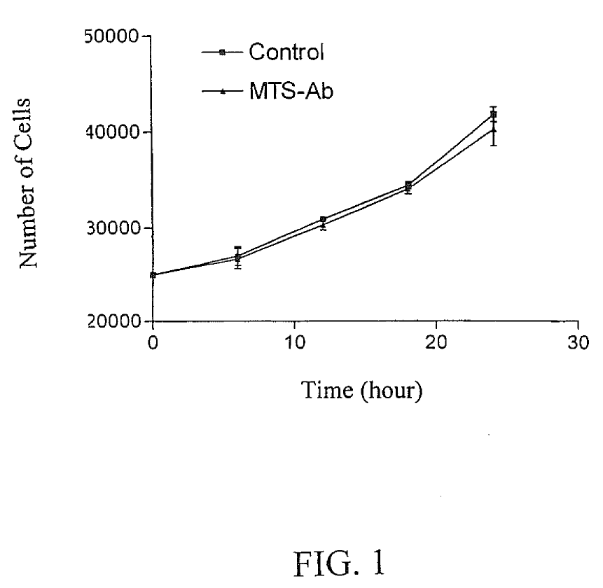

MTS Conjugated Anti-active Caspase-3 Antibody Shows Little Cell Growth Inhibition. First tested was the potential toxicity of MTS-antibody conjugates to the cells. The cell viability assay showed that the MTS-antibody conjugate exerted little effect on cell growth (FIG. 1).

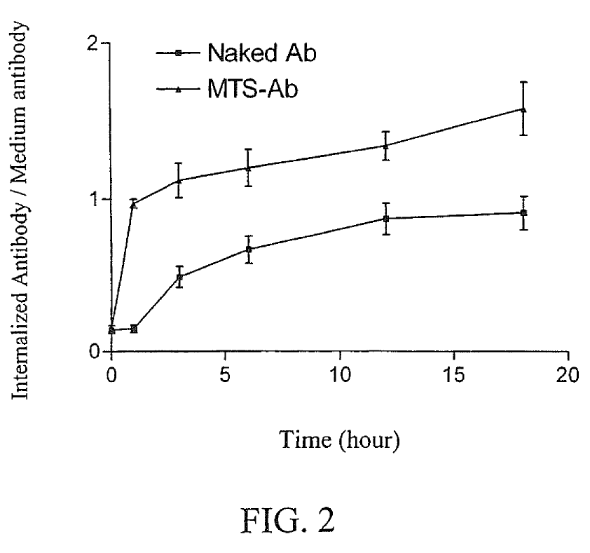

MTS Peptide Promotes Rapid Penetration of Anti Active Caspase-3 Antibody into Living Cells. The ELISA was designed to capture rabbit Ig using a sandwich assay. As seen in FIG. 2, the MTS conjugation rapidly promoted monoclonal anti-active caspase-3 antibody to internalize into the live cells. The translocation of Ig increased within 1 h and reached a plateau after 18 h. The antibody remaining in the culture decreased at 1 h and seemed to reach an equivalence at 18 h. The internalization of naked antibody was delayed (at 3 h) and remained at a lower level compared with MTS-conjugated anti-caspase-3 antibody.

Polyclonal MTS-anti Active Caspase-3 Antibody Inhibits DNA Fragmentation. MTS-conjugated or naked polyclonal anti-caspase-3 antibody (1 .mu.g/ml final concentration—equal to 1:64 dilution) was added to 6-ml cultured Jurkat cells and pre-incubated for 1 h. The antibody was washed out by centrifugation, fresh medium containing only actinomycin D (1 .mu.g/ml) without antibody was added, and cells were incubated for 4 h. Five ml of the culture was collected for DNA fragmentation. Naked (unconjugated) anti-caspase-3 polyclonal antibody did not prevent DNA laddering upon actinomycin D treatment. In contrast, MTS-conjugated anti-caspase-3 polyclonal antibody significantly inhibited DNA fragmentation (apoptosis) induced by actinomycin D (data not shown).

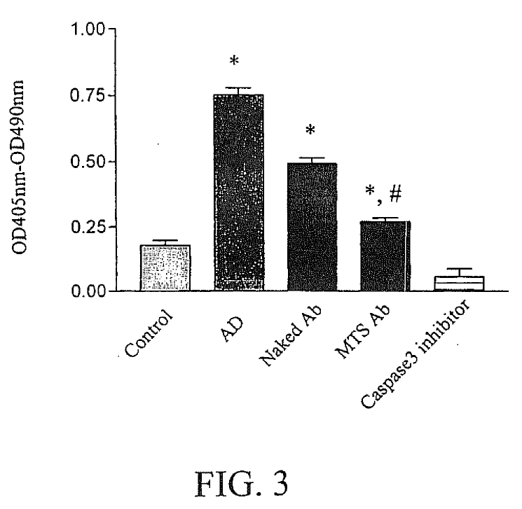

Monoclonal MTS-Anti Active Caspase-3 Antibody Prevents DNA Fragmentation. MTS-conjugated or naked monoclonal anti-caspase-3 antibody (1 .mu.g/ml final concentration) was added to 6-ml cultured Jurkat cells and pre-incubated for 1 h. The antibody was washed out by centrifugation, fresh medium containing actinomycin D (1 μg/ml) without antibody was added, and cells were incubated for 4 h. Five ml of the culture was collected for DNA fragmentation and the rest saved for Cell Death ELISA assay. MTS-conjugated antibody was observed to suppress DNA ladder formation while naked (unconjugated) anti-caspase-3 monoclonal antibody did not prevent DNA laddering upon actinomycin D treatment (data not shown). The Cell Death ELISA assay (FIG. 3) confirmed a significant decrease of cell apoptosis when cells are pre-treated with MTS-conjugated antibody. Jurkat cells incubated with caspase-3 inhibitor (DEVD-fmk), maintained 100% viability, and vehicle (DMSO)-treated control cells maintained about 80% viability. In the naked anti-caspase-3 antibody treatment group, only .about.36% of cells remained viable after 4 h. However, the MTS-anti-caspase-3 conjugated antibody treatment dramatically protected against actinomycin D induced apoptosis, as 70% of the cells remained viable (see Table 1).

TABLE 1*

| Treatment | % Viability - Exp. 1 | % Viability - Exp. 2 |
|---|---|---|
| None | 81.6 | 84.4 |
| AD | 18.0 | 24.0 |
| Naked 3H1 + AD | 24.5 | N.D. |
| MTS-3H1 + AD | 28.6 | N.D. |
| Naked anti-caspase-3 + AD | 37.4 | 34.4 |
| MTS-anti-caspase-3 + AD | 73.8 | 65.7 |

*None = cell culture medium with <0.2% DMSO; AD = 1 h actmomycin D treatment; 3H1 = control antibody; anti-caspase-3 = rabbit monoclonal anti-caspase 3 antibody. Apoptosis was detected using the cell death ELISA assay. The difference of ELISA readings between AD treatment and caspase-3 inhibitor (DEVD-fmk) treatment was judged as 100% viable. Exp. = experiment; N.D. = not done.

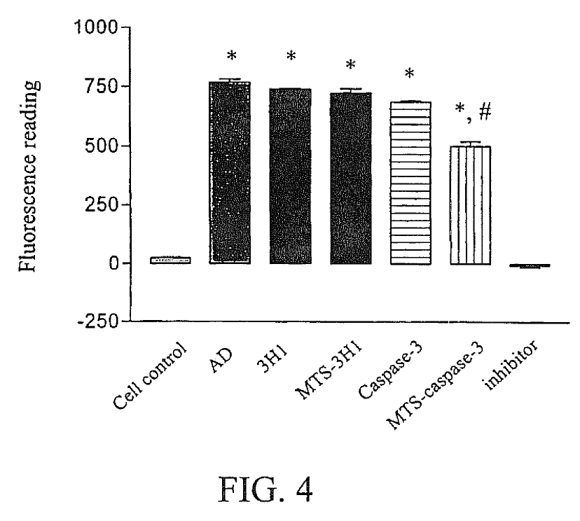

MTS-conjugated Anti Active Caspase-3 Antibody Suppresses Caspase-3 Activity. The Jurkat cells were treated similarly as in the previous section, and a murine anti-CEA antibody was modified and used as control. As shown in FIG. 4, caspase-3 like cleavage activity was increased upon actinomycin D treatment, MTS-conjugated monoclonal anti-active caspase-3 antibody reduced caspase-3 like cleavage activity, while the MTS-3H1 antibody showed no effect. Cell death ELISA assay also confirmed MTS-conjugated monoclonal anti-caspase-3 antibody showed significantly reduced DNA fragmentation (data not shown).

MTS-anti Active Caspase-3 Antibody Inhibits Spectrin Cleavage. As a downstream target of caspase-3, the protein levels of spectrin were examined. Two cleaved fragments of spectrin were observed in actinomycin D treated Jurkat cells (data not shown). Neither 3H1 nor MTS-3H1 protected spectrin from cleavage. Naked monoclonal anti-active caspase antibody showed little effect on protection; whereas MTS-conjugated anti-active caspase-3 antibody completely suppressed the cleavage of 100 kDa and 75 kDa alpha II spectrin fragments, as did caspase-3 inhibitor DEVD-fmk. The 150 kDa cleavage band showed no overt change in all antibody-pretreated cell samples.

### CONCLUSION

The above results indicate that anti-caspase-3 antibodies can inhibit significantly in-vitro apoptosis related events such as caspase-3 activity, DNA fragmentation, and spectrin cleavage. Anti-caspase-3 antibodies therefore can be utilized to inhibit apoptosis in a variety of diseases. In contrast to therapeutically used antibodies, conventional peptide apoptosis inhibitors exert strong inhibition but also have negative side effects as high toxicity, as shown in rodent animal models. Therefore, transport membrane-linked antibodies have a lower toxicity compared to conventional apoptosis inhibitors. Transport-membrane (MTS)-linked antibodies, therefore, represent promising new candidates for the treatment of diseases involving apoptosis, in particular, in the central nervous system for diseases such as Alzheimer's, Huntington's and Parkinson's.

The compositions of the invention are useful in pharmaceutical compositions for systemic administration to humans and animals in unit dosage forms, sterile solutions or suspensions, sterile non-parenteral solutions or suspensions oral solutions or suspensions, oil in water or water in oil emulsions and the like, containing suitable quantities of an active ingredient. Topical application can be in the form of ointments, creams, lotions, jellies, sprays, douches, and the like. The compositions are useful in pharmaceutical compositions (wt %) of the active ingredient with a carrier or vehicle in the composition in about 1 to 20% and preferably about 5 to 15%.

The above parenteral solutions or suspensions may be administered transdermally and, if desired a more concentrated slow release form may be administered. The cross-linked peptides of the invention may be administered intravenously, intramuscularly, intraperitoneally or topically. Accordingly, incorporation of the active compounds in a slow release matrix may be implemented for administering transdermally. The pharmaceutical carriers acceptable for the purpose of this invention are the art known carriers that do not adversely affect the drug, the host, or the material comprising the drug delivery device. The carrier may also contain adjuvants such as preserving stabilizing, wetting, emulsifying agents and the like together with the penetration enhancer of this invention. The effective dosage for mammals may vary due to such factors as age, weight activity level or condition of the subject being treated. Typically, an effective dosage of a compound according to the present invention is about 10 to 500 mg, preferably 2-15 mg, when administered by suspension at least once daily. Administration may be repeated at suitable intervals.

The purpose of the above description and examples is to illustrate some embodiments of the present invention without implying any limitation. It will be apparent to those of skill in the art that various modifications and variations of the compositions and methods of the present invention can be practiced within the scope of the appended claims without departing from the spirit or scope of the invention. All patents and publications cited herein are incorporated by reference in their entireties.

What is claimed is:

1. A compound effective in regulating normal or infected cell function, which compound comprises an antibody, or fragment thereof, conjugated to a membrane transporter peptide, which antibody, or fragment thereof, is immunospecific for: (a) a signaling protein internal a cell selected from the group consisting of caspases, kinases, and phosphatases, (b) an immature viral protein, (c) a cell-surface or intracellular tumor antigen, (d) a nuclear or nucleolar protein participating in regulation of DNA synthesis and gene expression, or (e) a cytoskeletal protein participating in cell proliferation or cytostasis.

2. The compound of claim 1, wherein the antibody is a monoclonal antibody.

3. The compound of claim 1, which is effective in inhibiting apoptosis and comprises an anti-caspase antibody, or fragment thereof, conjugated to a membrane transporter peptide.

4. The compound of claim 3, wherein the antibody is an anti-caspase-3 antibody.

5. The compound of claim 1, wherein the membrane transporter peptide is a translocation sequence (MTS) peptide.

6. The compound of claim 5, wherein the MTS peptide is endogenous to Kaposi fibroblast factor, TAT peptides of HIV-1, antennapedia homeodomain-derived peptide, herpes virus protein VP22, or transportan peptide.

7. The compound of claim 6, wherein the MTS peptide comprises the amino acid residue sequence AAVLLPVLLAAP (SEQ ID NO: 9).

8. The compound of claim 7, wherein the MTS peptide comprises the amino acid residue sequence KGEGAAVLLPVLLAAPG (SEQ ID NO: 8).

9. The compound of claim 1, wherein the membrane transporter peptide has reduced hydrophobicity relative to a second peptide containing the amino acid residue sequence: KGEGAAVLLPVLLAAPG (SEQ ID NO: 8), which membrane transporter peptide affords greater potentiation of internalization and immunoconjugate potency relative to the second peptide.

10. A pharmaceutical composition effective in inhibiting apoptosis in a human comprising an anti-caspase antibody, or fragment thereof, conjugated to a membrane transporter peptide.

11. The composition of claim 10, wherein the antibody is a monoclonal antibody.

12. The composition of claim 10, wherein the antibody is an anti-caspase-3 antibody.

13. The composition of claim 10, wherein the membrane transporter peptide is a membrane translocation sequence (MTS) peptide.

14. The composition of claim 10, wherein the MTS peptide comprises the amino acid residue sequence AAVLLPVLLAAP (SEQ ID NO: 9).

15. The composition of claim 14, wherein the MTS peptide comprises the amino acid residue sequence KGEGAAVLLPVLLAAPG (SEQ ID NO: 8).

16. A method of treating or preventing a disease in humans comprising administering to a patient in need thereof a pharmacologically effective amount of a composition comprising an anti-caspase antibody, or fragment thereof conjugated to a membrane transporter peptide.

17. The method of claim 16, wherein the disease is Alzheimer's disease, Huntington's disease, or Parkinson's disease.


SEQUENCE LISTING (14 sequences)

SEQ ID NO: 1 - protein, 16 aa, Artificial Sequence
  Synthesized peptide with sequence derived from position 1217-1232
KNRWEDPGKQLYNVEA

SEQ ID NO: 2 - DNA, 84 bp, Artificial Sequence
  Synthesized oligonucleotide
gatcgcagccgttcttctccctgttcttcttgccgcacccggcgtcggcaagaagagggacaagaagaacggcgtgggccctag

SEQ ID NO: 3 - DNA, 95 bp, Artificial Sequence
  Synthesized oligonucleotide
gatccccgcagccgttcttctccctgttcttcttgccgcaccctagcgggcgtcggcaagaagagggacaagaagaacggcgtgggattcgctag

SEQ ID NO: 4 - DNA, 40 bp, Artificial Sequence
  Synthesized oligonucleotide primer for Grb2 SH2 cDNA
ccggatccccgaaatgaaaccacatccgtggttttttggc

SEQ ID NO: 5 - DNA, 39 bp, Artificial Sequence
  Synthesized oligonucleotide primer for Grb2 SH2 cDNA
ccggatcccgagggcctggacgtatgtcggctgctgtgg

SEQ ID NO: 6 - DNA, 40 bp, Artificial Sequence
  Synthesized oligonucleotide primer for Stat1 SH2 cDNA
ccggatccccaaacacctgctccctctctggaatgatggg

SEQ ID NO: 7 - DNA, 38 bp, Artificial Sequence
  Synthesized oligonucleotide primer for Stat1 SH2 cDNA
ccggatccctctagagggtgaacttcagacacagaaat

SEQ ID NO: 8 - protein, 17 aa, Artificial Sequence
  Synthesized peptide
KGEGAAVLLPVLLAAPG

SEQ ID NO: 9 - protein, 12 aa, Artificial Sequence
  Synthesized peptide
AAVLLPVLLAAP

SEQ ID NO: 10 - protein, 16 aa, Artificial Sequence
  Synthesized peptide
RQIKIWFQNRRMKWKK

SEQ ID NO: 11 - protein, 7 aa, Artificial Sequence
  Synthesized peptide
RRMKWKK

SEQ ID NO: 12 - protein, 27 aa, Artificial Sequence
  Synthesized peptide
GWTLNSAGYLLGKINLKALAALAKKIL

SEQ ID NO: 13 - protein, 21 aa, Artificial Sequence
  Synthesized peptide
AGYLLGKINLKALAALAKKIL

SEQ ID NO: 14 - protein, 4 aa, Artificial Sequence
  Synthesized peptide
KLAL